(19) United States
(12) Patent Application Publication
(10) Pub. No.: US 2010/0048759 A1
(43) Pub. Date: Feb. 25, 2010

(54) METHOD FOR LUBRICATING SURGICAL INSTRUMENTS

(75) Inventor: Kim R. Smith, Woodbury, MN (US)

Correspondence Address: IPLM GROUP, P.A., POST OFFICE BOX 18455, MINNEAPOLIS, MN, 55418, US

(73) Assignee: Ecolab Inc.

(21) Appl. No.: 12/197,153

(22) Filed: Aug. 22, 2008

(51) Int. Cl. A61L 31/10

(52) U.S. Cl. 523/105

(57) ABSTRACT

Surgical instruments may be lubricated by contacting them with a lubricious, water-soluble lubricating composition containing a blend of glycerin and polyethylene glycol. The composition can provide excellent lubrication while still allowing steam to contact the instrument surface. The composition may be provided as substantially surfactant-free blend at least 50 wt. % of which is glycerin and polyethylene glycol. The blend may be placed in a suitable dispenser and exposed to water to form the lubricating composition.

### TECHNICAL FIELD

This invention relates to surgical instrument lubrication.

### BACKGROUND

Instrument baths (also referred to as milkbaths) are oil/water emulsions used in healthcare to lubricate hinges and other moving or sliding parts in surgical instruments. They take their name from the appearance of their diluted use solution, which is usually an emulsion having an appearance very similar to milk. Recent developments in instrument bath formulation include a switch from mineral oil-based emulsions to emulsions based on vegetable oils or vegetable oil derivatives. Vegetable oil or vegetable oil derivative emulsions may provide opaque or clear ready-to-use (RTU) solutions. Other recent developments include a change from bath to spray application so as to reduce the risk that bacteria or other organisms will be transferred between instruments.

Commercial instrument lubricants include ASEPTI-LUBE™ and ASEPTI-MB™ lubricants, both from the Healthcare Division of Ecolab Inc.

### SUMMARY OF THE INVENTION

The oils in some instrument lubricants may prevent or inhibit proper steam sterilization due to reduced wetting caused by residual oil remaining on the instrument surface. The present invention provides, in one aspect, a method for lubricating a surgical instrument, which method comprises the step of contacting the instrument with a lubricious, water-soluble lubricating composition comprising a blend of glycerin and polyethylene glycol (PEG). The composition may be liquid, gel, paste or solid. The composition preferably is free or substantially free of oil(s). The composition can provide excellent lubrication while still allowing steam to contact the instrument surface.

The invention provides, in another aspect, a surgical instrument lubricant composition comprising a substantially surfactant-free blend of glycerin and polyethylene glycol wherein the glycerin and polyethylene glycol represent at least about 50 wt. % of the composition. The blend may be placed in a suitable dispenser and exposed to water to form the above-mentioned lubricating composition.

### BRIEF DESCRIPTION OF THE DRAWING

FIG. 1 is a perspective view of a hinged surgical instrument being spray-lubricated using the disclosed method;

FIG. 2 is a perspective view of a package containing a water-soluble shaped solid block of the disclosed surgical instrument lubricant concentrate; and

FIG. 3 is a graph showing the force required to open a pair of scissors lubricated using a variety of lubricants.

Like reference symbols in the various figures of the drawing indicate like elements. The elements in the drawing are not to scale.

### DETAILED DESCRIPTION

The following detailed description describes certain illustrative embodiments and is not to be taken in a limiting sense. All weights, amounts and ratios herein are by weight, unless otherwise specifically noted.

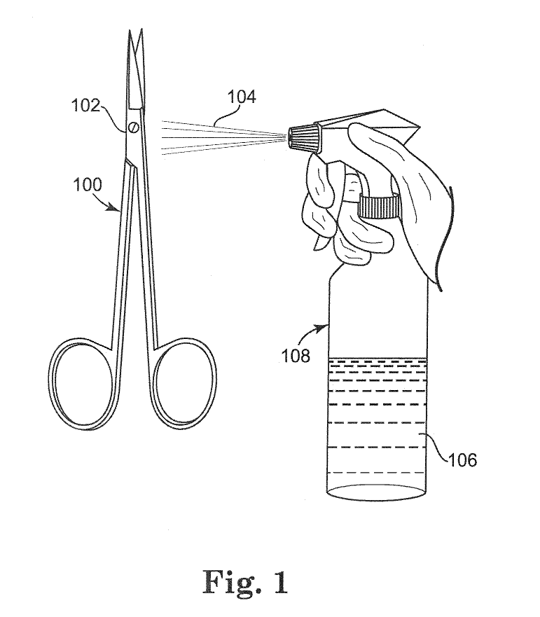

Referring to FIG. 1, an instrument such as surgical scissors 100 may be lubricated by applying onto at least hinge portion 102 a mist 104 of liquid lubricating composition 106 dispensed from spray applicator 108. Applicator 108 may be a trigger sprayer, pressure sprayer or any other convenient device for applying composition 106 to a surgical instrument. A variety of other application techniques or devices may be employed. For example, when the composition is in liquid form an instrument may be immersed in the composition (e.g., in a bath), or a stream or drops of the composition may be directed towards, sponged upon or otherwise allowed to contact the instrument (e.g., in an instrument washer). When the composition is in solid form, instruments may be rubbed or otherwise contacted with the composition so as to transfer some of the composition to the instruments. Normal room temperature solid forms of the composition may also be heated to convert them to liquids prior to contact with instruments.

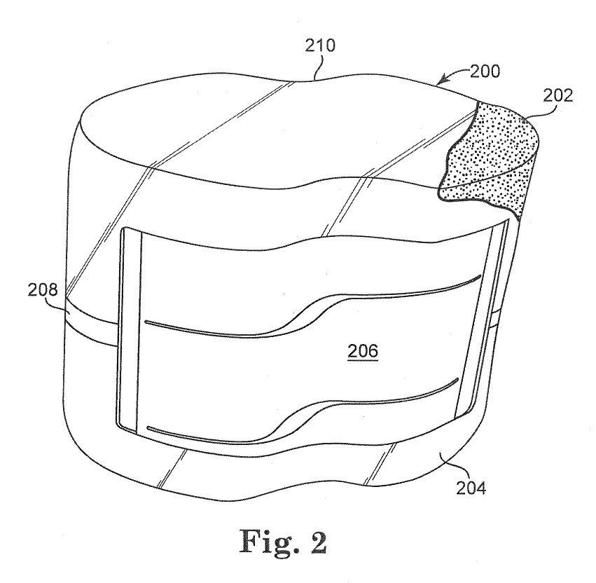

FIG. 2 shows a perspective view of a package 200 containing a water-soluble shaped solid block 202 of the disclosed surgical instrument lubricant concentrate. Block 202 is surrounded by wrapping 204 made of plastic, paper or other suitable material. Label 206 provides a user with required or desirable information regarding package 200. Weakened tear line or fracture line 208 incorporated in wrapping 204 facilitates removal of block 202 from its wrapping. Block 202 may be shaped into any suitable profile using casting, extrusion or other suitable shaping techniques. The shape shown in FIG. 2 has a generally elliptical profile with a pinched waist 210 whose shape desirably matches that of a corresponding receptacle in a suitable dispenser (not shown in FIG. 2) that may combine block 202 with water so that the resulting solution may be brought into contact with a surgical instrument. By selecting a suitable shape for block 202 and the corresponding receptacle, a manufacturer can reduce the likelihood that the dispenser will be refilled with the wrong concentrate or with a contaminated concentrate. A variety of dispensers and a variety of shapes may be employed, for example those shown in U.S. Pat. Nos. 4,687,121, 4,826,661, 5,086,952, 5,389,344, 6,143,257 and 6,489,278.

A variety of surgical instruments may be lubricated using the disclosed lubricating composition. Exemplary instruments include scissors, shears, forceps, pliers, hemostats, clamps, specula, retractors, spreaders, separators, cuffs, positioning tools, insertion tools, scalpels, tubing, catheters, drainage tubes, stents, syringes, needles, pipettes, collection tubes, wires, probes, irrigators, aspirators, gloves, staples, sutures, and the like. The instruments may have sliding surfaces or other moving parts which contact other parts of the instrument or other surfaces including tissue. The lubricated surface may exhibit various benefits as a result of the lubrication including improved lubricity (viz., reduced friction), improved durability or increased abrasion resistance. The lubricated surface may be made from a variety of materials including metals, alloys, plastics including polymers, glass or ceramics. Representative metals and alloys include stainless steel, nickel, nickel-cobalt, NiTinol, gold, platinum, silver, tantalum, titanium, tungsten, rare earth metals, combinations thereof and alloys and plated articles thereof. Representative plastics include polyacrylates and polymethacrylates including polymethylmethacrylate, polymethylacrylate and polybutylmethacrylate; polyamides including nylon polymers such as nylon-11 and nylon-12; polycarbonates; polyesters including polyethylene terephthalate (PET); polyethers; polyimides; polyolefins including polyethylenes (PE), polypropylenes (PP) and polybutadienes; polystyrene; polyvinyl acetate; polyvinyl chloride; polynrethanes; natural and synthetic rubbers; silicones including silicone rubbers; and combinations and copolymers thereof. Exemplary polymers or copolymers include styrene-butadiene copolymers; ethylene-propylene copolymers, styrenic block copolymers (e.g., KRATON™ polymers from Kraton Polymers LLC); block copolymers of polyether and polyester polymers (e.g., HYTREL™ copolymers from DuPont Plastics); block copolymers of polyethers and polyamides (e.g., PEBAX™ resins from Arkema Inc.); synthetic hydrocarbon copolymers including styrene-butadiene rubber (SBR) and ethylene propylene diene monomer rubber (EPDM); and fluorocarbon polymers and copolymers including polyvinylidene fluoride (PVDF), polytetrafluoroethylene (PTFE) and polyperfluoroethylenepropylene (FEP).

Glycerin for use in the disclosed lubricating compositions may be obtained from a variety of sources, for example as a byproduct from epichlorohydrin production or from biodiesel manufacture. USP grade glycerin is preferred, but other grades may be employed if desired.

Polyethylene glycols (PEGs) for use in the disclosed lubricating compositions may be obtained from variety of sources, including the PLURACOL™ line of PEGs from BASF. Mixtures of PEGs may be employed. The chosen PEGs may for example be liquids or solids.

The glycerin and PEG may be combined in a wide range of relative amounts to form the disclosed concentrate, for example at a glycerin:PEG ratio of about 1:9-9:1 or about 1:7-7-1. The PEG molecular weight may be selected to provide a mixture with an appropriate physical form, e.g., a liquid, gel, paste or solid, with PEGs that are normally liquid at room temperature generally being used to provide liquid lubricant compositions, and PEGs that are normally solid at room temperature generally being used to provide solid lubricant compositions. The PEG may be linear or branched and may for example have a number average molecular weight of 106 to about 20,000 g/mole, about 148 to about 10,000 g/mole or about 236 to about 8,000 g/mole, with molecular weights less than about 2,000 likely to provide liquid concentrates and molecular weights greater than about 2,000 likely to provide solid concentrates. Water may be incorporated into the concentrate, for example as a processing aid to modify the viscosity or melting point. The water may be tap water, softened water, deionized water, distilled water or water in any other suitable form. Water may represent for example at least 5 wt. %, at least 10 wt. %, at least 20 wt. %, at least 25 wt. %, at least 30 wt. %, at least 40 wt. %, at least 50 wt. % or at least 60 wt. % of an RTU solution or concentrate. Water may for example also represent less than 80 wt. %, less than 70 wt. %. less than 60 wt. %, less than 55 wt. %, less than 50 wt. %, less than 40 wt. %, less than 30 wt. % or less than 20 wt. % of an RTU solution or concentrate. The RTU solution or concentrate may also be substantially water-free.

The RTU solution or concentrate may include a variety of other adjuvants to alter its performance or properties before or after contact with a surgical instrument. Useful adjuvants include antioxidants, anticorrosion additives, antimicrobial agents, antirust additives, cosolvents, coalescents, colorants, defoamers, dyes, fragrances, indicators, light stabilizers, preservatives, and water-dispersible opacifying agents (for example, PEG-stearates). The types and amounts of such adjuvants will be apparent to those skilled in the art. Surfactants may also be employed, but the composition desirably is substantially free of surfactants. The disclosed RTU solutions and concentrates may comprise glycerin, PEG and one or more of any of the adjuvants referred to above, or may consist essentially of or consist of glycerin, PEG and one or more adjuvants. Preferably such adjuvants are on the U.S. Food and Drug Administration (FDA) list of substances (see 21 C.F.R Part 184) affirmed by the FDA as being Generally Recognized as Safe (GRAS) for use as direct food ingredients. Preferably the combined weight of glycerin and PEG represents at least about 50 wt. %, at least about 60 wt. % or at least about 70 wt. % of the disclosed RTU solution or concentrate. The RTU solutions or concentrate may be made from sterile ingredients, sterilized at the time of packaging, or sold in unsterilized form.

Use solutions of the composition may be clear, translucent or opaque, and may for example be made at concentrate:water ratios of about 1:500-1:10, or about 1:250-1:1. The desired use concentration may for example vary somewhat depending on the desired application method (manual spray, soak, instrument washer, etc.). The composition may for example be applied to an instrument surface to be lubricated and the excess wiped away, rinsed off, or removed during a steam sterilization cycle.

The disclosed lubricants may be used for other purposes, for example as a food-grade conveyor lubricant for use in beverage plants.

The invention is further illustrated in the following non-limiting examples.

### EXAMPLE 1

### Preparation of Solid Concentrate

A solid, oil-free instrument bath concentrate was prepared by heating 25 parts glycerin to 66° C. and adding 75 parts PEG-8000 (8000 molecular weight) to the melt. The resulting molten mixture was transferred to a plastic container and allowed to cool to ambient temperature. A shaped hard solid block was obtained.

### EXAMPLE 2

### Preparation of Liquid Concentrate

A liquid, oil-free instrument bath concentrate was prepared by mixing together 25 parts glycerin and 75 parts PEG-400 (400 molecular weight) to afford a clear liquid mixture.

### EXAMPLE 3

### Comparison of Solid Concentrate and Conventional Oil-based Milkbath

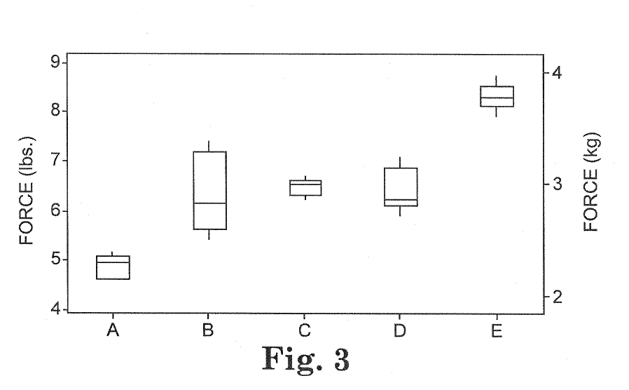

Dilutions of the Example 1 concentrate were prepared at 1:128 and 1:6 concentrate:water ratios using water with 5 grain hardness. A commercial oil-based milkbath, ASEPTI MB Instrument Lubricant, was similarly diluted. A pair of surgical scissors was immersed in each solution and the pounds of force required to open the scissors measured using a strain gauge in a 5-fold replicate experiment. Between test solutions, the scissors were cleaned thoroughly with acetone and dried to remove residual lubricant. The results are shown in FIG. 3, with bars A through E respectively showing the range and average force values obtained for the Example 1 concentrate at 1:128 dilution (bar A), the commercial concentrate at 1:128 dilution (bar B), the Example 1 concentrate at 1:6 dilution (bar C), the commercial concentrate at 1:6 dilution (bar D) and no lubricant (bar E). The lowest opening force was obtained when using the Example 1 concentrate at a 1:128 dilution. Based on their behavior when exposed to water drops, lubricants based on the Example 1 concentrate appeared to be sufficiently hydrophilic to render the scissors readily amenable to steam sterilization.

Various modifications and alterations of this invention will be apparent to those skilled in the art without departing from the scope of this invention. It should be understood that this invention is not limited to the illustrative embodiments set forth above.

We claim:

1. A method for lubricating a surgical instrument, which method comprises the step of contacting the instrument with a lubricious, water-soluble lubricating composition comprising a blend of glycerin and polyethylene glycol.

2. A method according to claim 1 wherein the composition is a liquid.

3. A method according to claim 1 wherein the composition is a gel or paste.

4. A method according to claim 1 wherein the composition is a solid.

5. A method according to claim 1 wherein the composition is substantially free of surfactants.

6. A method according to claim 1 wherein the composition is substantially free of oils.

7. A method according to claim 1 wherein the composition is substantially free of water.

8. A method according to claim 1 wherein the composition provides a hydrophilic surface on the instrument.

9. A method according to claim 1 wherein the composition lubricates the instrument and promotes contact by steam with the instrument surface.

10. A method according to claim 1 wherein the instrument comprises a scissors, shears, forceps, pliers, hemostat, clamp, speculum, retractor, spreader, separator, cuff, positioning tool, insertion tool, scalpel, tubing, catheter, drainage tube, stent, syringe, needle, pipette, collection tube, wire, probe, irrigator, aspirator, glove, staple or suture.

11. A method according to claim 1 wherein the instrument has a sliding surface or other moving part which contacts other parts of the instrument.

12. A method according to claim 1 wherein the instrument has a sliding surface or other moving part which contacts tissue.

13. A method according to claim 1 wherein the glycerin and polyethylene glycol represent at least about 50 wt. % of the composition.

14. A method according to claim 1 wherein the glycerin and polyethylene glycol represent at least about 60 wt. % of the composition.

15. A method according to claim 1 wherein the glycerin and polyethylene glycol represent at least about 70 wt. % of the composition.

16. A surgical instrument lubricant composition comprising a substantially surfactant-free blend of glycerin and polyethylene glycol wherein the glycerin and polyethylene glycol represent at least about 50 wt. % of the composition.

17. A composition according to claim 16 wherein the glycerin and polyethylene glycol represent at least about 60 wt. % of the composition.

18. A composition according to claim 16 wherein the glycerin and polyethylene glycol represent at least about 70 wt. % of the composition.

19. A composition according to claim 16 in the form of a liquid.

20. A composition according to claim 16 in the form of a gel or paste.

21. A composition according to claim 16 in the form of a solid.

22. A composition according to claim 21 in the form of a water-soluble shaped solid block.

23. A composition according to claim 16 wherein the composition is substantially free of oils.

24. A composition according to claim 16 wherein the composition is substantially free of water.

25. Q composition according to claim 16 wherein the composition consists essentially of glycerin, polyethylene glycol and one or more adjuvants on the U.S. Food and Drug Administration list of substances affirmed as being Generally Recognized as Safe for use as direct food ingredients.
